SEQUENCE LISTING (4 sequences)

SEQ ID NO: 1 - DNA, 19 bp, Artificial Sequence
  Artificially synthesized oligonucleotide primer
ctgcgtctggacctggctg

SEQ ID NO: 2 - DNA, 19 bp, Artificial Sequence
  Artificially synthesized oligonucleotide primer
acgcggcagtggccatctc

SEQ ID NO: 3 - DNA, 18 bp, Artificial Sequence
  Artificially synthesized oligonucleotide primer
ccatgccgagagtgatcc

SEQ ID NO: 4 - DNA, 18 bp, Artificial Sequence
  Artificially synthesized oligonucleotide primer
gaagcgcgatcacatggt

(19) United States
(12) Patent Application Publication
(10) Pub. No.: US 2021/0236336 A1
(43) Pub. Date: Aug. 5, 2021

(54) APPLICATOR FOR CORNEAL THERAPEUTICS

(71) Applicant: North Carolina State University, Raleigh, NC (US)

(72) Inventors: Brian C. Gilger, Raleigh, NC (US); Vladimir Zarnitsyn, Atlanta, GA (US); Samirkumar Patel, Marietta, GA (US)

(21) Appl. No.: 17/053,923

(22) PCT Filed: May 9, 2019
(86) PCT No.: PCT/US2019/031518

§ 371 Date: Nov. 9, 2020

Related U.S. Application Data
(60) Provisional application No. 62/668,975, filed on May 9, 2018.

(51) Int. Cl. A61F 9/00; A61M 37/00

(52) U.S. Cl. CPC A61F 9/0017; A61M 2202/064; A61M 37/0084

(57) ABSTRACT

A corneal injection needle is disclosed. The corneal injection needle includes a shaft, a housing, and a stop feature for controlling the depth of the needle penetration. The needle is capable of delivering precise amounts of injectable material to the cornea with low leakage rates to off-target areas and less corneal damage compared to conventional needles.

### RELATED APPLICATIONS

This application claims priority to and the benefit of U.S. Provisional Patent Application Ser. No. 62/668,975, filed on May 9, 2018, the disclosure of which is incorporated herein by reference in its entirety.

### TECHNICAL FIELD

The presently disclosed subject matter relates to apparatuses and methods for contacting target areas of the eye, including delivering agents to the eye, including in particular to the cornea.

### BACKGROUND

As treatments develop for corneal disease, precise application of low volumes of therapeutics is increasingly important to limit off target treatment effects and to maximize desired therapy. Treatment of specific locations of the cornea, such as the epithelium, stroma, or endothelium, is particularly important when using novel therapies, such as gene or cell therapy. Currently, treatment of the cornea is largely relegated to use of eye drops, which are very inefficient (<10% of drug enters the cornea) and target either the whole cornea or the ocular surface, not precise anatomic locations. Also, the standard techniques of ocular injections result in perforation of the eye, and thus increased complication. Thus, there is a present and ongoing need in the art for improved approaches for corneal-based therapy and/or other therapy of the eye.

### SUMMARY

In some embodiments the presently disclosed subject matter provides a device configured for selectively contacting a target region of the eye of a subject. In some embodiments, the target region is selected from the group consisting of the cornea, an ocular anterior segment, the conjunctiva, an anterior chamber, iridocorneal angle, trabecular meshwork, sclera, subretinal space, choroid, and Schlem's canal. In some embodiments, a configuration of the device for selectively contacting a target region of the eye varies based on a patient species, an intended agent to be delivered, and/or an intended tissue target in the target region.

In some embodiments, the device comprises a shaft, the shaft having a first end and a second opposite end having a tip. In some embodiments, a hollow pathway is disposed between the first end and the second end. In some embodiments, the tip has a dimension configured for selectively contacting the target region of the eye. In some embodiments, a hub houses the shaft, the hub having a proximal end and a distal end. In some embodiments, the tip comprises a bevel having a dimension configured for selectively contacting the target region of the eye. In some embodiments, the hub comprises a connector at the proximal end for connecting to a delivery device. in some embodiments, the hub has a predetermined shape configured based on the target region of the eye, optionally wherein the hub shape comprises a bullnose, further optionally wherein the bullnose has a radius of curvature of about 0.2 mm to about 1.5 mm or optionally wherein the hub shape comprises a conical shape, further optionally wherein the conical shape comprises a cone angle ranging between about 45 and about 150 degrees.

In some embodiments, a stop region is disposed at the distal end of the hub such that only the tip of the shaft extends beyond the stop region. In some embodiments, the stop region comprises a biocompatible elastomeric material, optionally wherein the elastomeric material comprises silicone. In some embodiments, the shaft comprises a metal, optionally wherein the metal is stainless steel, further optionally wherein the stainless steel is an alloy 304 or 310 material.

In some embodiments, the hub comprises a polymer, optionally wherein the polymer is selected from the group consisting of polypropylene (PP), polyethylene (PE), polyether ether ketone (PEEK), acrylonitrile butadiene styrene (ABS). in some embodiments, the hub comprises a surface, wherein the surface comprises or is coated by biocompatible elastomer, optionally wherein the biocompatible polymer comprises a silicone.

In some embodiments, the length of the shaft tip and/or the length of the bevel is less than a thickness of the target region of the eye. In some embodiments, the target region is the cornea and the length of the tip and/or the length of the bevel is less than the thickness of the cornea. In some embodiments, the length of the tip ranges from about 0.1 to about 1 mm. In further embodiments, the length of the bevel is less than the length of the tip, optionally wherein the length of the bevel ranges from about 0.1 to about 0.8 mm. In other embodiments, the dimension of the tip comprises a gauge and the gauge of the tip ranges from about 30 gauge to about 40 gauge. In further embodiments, the tip comprises an opening and the device is configured to deliver an injection volume, optionally wherein the injection volume ranges from about 1 to about 200 microliters.

In some aspects, a syringe is connected to the hub of the device. In some embodiments, the syringe comprises a syringe pump. In some embodiments, the syringe comprises a reservoir containing an agent to be delivered. In some aspects, the device is provided in a system for selectively contacting a target region of the eye.

A method for selectively contacting a target region in the eye of a subject is also disclosed herein. The method comprises providing a targeting device as disclosed herein and contacting the target region of the eye with the device. In some aspects, contacting the target region of the eye comprises delivering an agent to the target region and/or facilitating a surgery on the target region of the eye. In some embodiments, the agent comprises a fluid or a powder. In some embodiments, the agent comprises a therapeutic agent, an imaging agent, an implant, an ink, and/or a surgical enhancement.

In some embodiments, the therapeutic agent is selected from the group consisting of a topical ocular medication, optionally wherein the topical ocular medication is selected from the group consisting of a steroid, an antibiotic, a NSAID, and an anti-glaucoma agent; a gene therapy vector, optionally wherein the gene therapy vector comprises a virus; a stem cell; and combinations thereof. In some embodiments, the imaging agent comprises gadolinium.

In other aspects, an implant comprises a micro-electronic, optionally wherein the micro-electronic comprises a visual aide or an intraocular pressure gauge. In further aspects, the ink comprises an ink for cosmetic or therapeutic tattooing. In yet further aspects, the surgical enhancement is a viscoelastic and/or a surgical enhancement for corneal surgery or Lasik surgery.

In some embodiments, the method is used where the target region comprises a diseased and/or injured region of the eye. In some embodiments, the diseased and/or injured region comprises an infected region of the eye. In some embodiments, the diseased and/or injured region is in the cornea. In some embodiments, the method comprises treating a disease selected from the group consisting of an infection, (for example, the infection is a corneal stromal infection); an immune-mediated disease; a genetic disease (for example, the genetic disease is MPS-1); a neovascular disease; and a degenerative disease.

In some embodiments, contacting comprises improving drug penetration or directly providing therapy for an ocular anterior segment disease and/or injury. In some embodiments, the ocular anterior segment disease and/or injury is uveitis, glaucoma, and/or trauma. In some embodiments, the contacting comprises positioning the device perpendicular to the target region in the eye of the subject.

In some embodiments, the device is used in conjunction with an imaging technique. In some embodiments, the imaging technique employs an optical coherence tomography device or a high frequency ultrasound, such that a characteristic of the target region to be contacted is determined and a device having a desired configuration is prepared and/or selected to reach the target region, optionally without passing through the target region. In some embodiments, the imaging technique determines the thickness and/or depth of the target region and allows the selection of an appropriately configured device to reach the target region.

In some embodiments, the device can be provided in a kit of parts for selectively contacting a target region of the eye, the kit of parts comprising one or more devices and a container for the devices.

Accordingly, it is an object of the presently disclosed subject matter to provide apparatuses and methods for administering therapeutic agents to the eye, including particularly to the cornea. This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description of the presently disclosed subject matter and non-limiting Figures and Examples.

### BRIEF DESCRIPTION OF THE DRAWINGS

FIG. 1 is an isometric view of an embodiment of a corneal injection needle as disclosed herein;

FIG. 2 is an assembly view of an embodiment of a corneal injection device as disclosed herein;

FIGS. 3A-3B are isometric views of another embodiment of a corneal injection needle as disclosed herein;

FIGS. 4A-6B are views illustrating a method of using a corneal injection needle as disclosed herein;

FIGS. 7A-7D are charts comparing test results from a conventional needle and a corneal injection needle as disclosed herein;

FIG. 8 is a chart comparing incision depths of a corneal injection needle as disclosed herein;

FIG. 9 is a chart of injected fluorescence presence over time using a corneal injection needle as disclosed herein;

FIGS. 10A-10B are charts comparing genome vectors applied with a corneal injection needle as disclosed herein;

FIG. 11 is a chart comparing test results from a conventional needle and a corneal injection needle as disclosed herein;

FIGS. 12A-12B are charts comparing ocular inflammation using a corneal injection needle as disclosed herein;

FIGS. 13A-13B are charts comparing visible fluorescence using a corneal injection needle as disclosed herein; and

FIG. 14 is a chart of genome density over time using a corneal injection needle as disclosed herein.

FIGS. 15A-15D are charts comparing voriconazole concentration following application of either 500 μg of topical voriconazole (divided into 4 doses, given every 6 hours), a single intrastromal injection of 500 μg voriconazole (using a PCI needle) or a single Intrastromal injection of saline in normal New Zealand white rabbits. FIG. 15A, tears; FIG. 15B, conjunctiva; FIG. 15C, aqueous humor; FIG. 15D, vitreous humor.

### DETAILED DESCRIPTION

The presently disclosed subject matter addresses obstacles to corneal therapy and/or other therapy of the eye. In some embodiments, precise application of low volumes of therapeutics is provided to limit off target treatment effects and to maximize desired therapy. In some embodiments, treatment of specific locations of the cornea, such as the epithelium, stroma, or endothelium, is provided, including but not limited to with gene or cell therapy. The presently disclosed subject matter also provides for precise anatomical treatment using low volumes of therapeutics. In some embodiments, precise imaging (e.g., optical coherence tomography) and an innovative therapeutic device provide for delivery of therapeutics easily, precisely, practically, and repeatedly. Furthermore, in the emerging field of corneal therapeutics, the presently disclosed subject matter can be used in any desired therapeutic or cosmetic applications.

The presently disclosed subject matter will now be described more fully hereinafter with reference to the accompanying Figures and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art. Certain components in the Figures are not necessarily to scale, emphasis instead being placed upon illustrating the principles of the presently disclosed subject matter (in some cases schematically).

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

While the following terms are believed to be well understood by one of ordinary skill in the art, the following definitions are set forth to facilitate explanation of the presently claimed subject matter.

Following long-standing patent law convention, the terms “a”, “an”, and “the” refer to “one or more” when used herein, including in the claims.

As used herein, the term “about”, when referring to a value or an amount, for example, relative to another measure, is meant to encompass variations of in some embodiments ±20%, in some embodiments ±10%, in some embodiments ±5%, in some embodiments ±1%, and in some embodiments ±0.1% from the specified value or amount, as such variations are appropriate. The term “about” can be applied to all values set forth herein.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed in some embodiments as a “p-value”. Those p-values that fall below a user-defined cutoff point are regarded as significant. In some embodiments, a p-value less than or equal to 0.05, in some embodiments less than 0.01, in some embodiments less than 0.005, and in some embodiments less than 0.001, are regarded as significant.

The term “clinical fluid” or “clinical sample” is used to include materials derived from animals or humans including but not limited to whole blood, serum, plasma, urine, tissue aspirates, saliva, mucous, and any other samples derived from living tissues.

In some embodiments, the subject treated according to the presently disclosed subject matter is a human subject, although it is to be understood that the methods described herein are effective with respect to all mammals.

More particularly, provided herein is the treatment of mammals, such as humans, as well as those mammals of importance due to being endangered (such as Siberian tigers), of economic importance (animals raised on farms for consumption or another use (e.g., the production of wool) by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Thus, embodiments of the methods described herein include the treatment of livestock and pets.

In some embodiments the presently disclosed subject matter provides a device configured for selectively contacting a target region of the eye of a subject.

In some embodiments, the device is configured to provide for selective injection of the target region of the eye. In some embodiments, the presently disclosed device is a precise injection device of appropriate size and configuration to allow delivery of small volumes of therapeutics (drugs, proteins, cells, gene therapy, etc.) to a target region of the eye. Examples of target regions of the eye include but are not limited to the cornea or ocular anterior segment (by way of additional example and not limitation, conjunctiva, cornea, anterior chamber, iridocorneal angle, trabecular meshwork, sclera, Schlem's canal, and/or subretinal space). In some embodiments, the device comprises a syringe connector or hub and a shaft having a tip, wherein the shaft and tip have a particular length and configuration to provide optimal delivery to the target region of the eye, such as but not limited to the cornea or ocular anterior segment (by way of additional example and not limitation, conjunctiva, cornea, anterior chamber, iridocorneal angle, trabecular meshwork, sclera, Schlem's canal, and/or subretinal space).

In some embodiments, the device comprises a hub and a shaft, the shaft having a first end and a second opposite end having a tip, optionally with a hollow pathway disposed between the first end and the tip at the second end; wherein the first end of the shaft is connected to the hub; wherein the tip has a dimension configured for selectively contacting the target region of the eye; and optionally wherein the tip has a bevel having a dimension configured for selectively contacting the target region of the eye. In some embodiments, the shaft comprises a needle, such as a microneedle. However, as disclosed herein, the configurations of the shaft, tip, and/or bevel afford more precision than a conventional microneedle, so as to provide for selectively contacting a target region of the eye as described herein.

In some embodiments, the target region of the eye is selected from the group comprising the cornea, an ocular anterior segment, the conjunctiva, an anterior chamber, iridocorneal angle, trabecular meshwork, sclera, subretinal space, choroid, and Schlem's canal. The term “target region” is meant to encompass any portion of a region of the eye, including a portion of the representative regions of the eye listed herein.

The cornea is the clear protective covering at the front of the eye. When light enters the cornea, it is refracted so that the rays pass freely through the pupil. The cornea is responsible for 65-75% of the eye's total focusing power. The cornea is made up of five layers: (1) epithelium, which blocks foreign material and absorbs oxygen and nutrients; (2) Bowman's membrane, which comprises collagen; (3) stroma, which aids in light conduction; (4) Descemet's membrane, which is a protective barrier; and (5) endothelium, which removes excess fluid. In some embodiments, the device shaft and tip of the shaft are configured to provide for selectively contacting a corneal layer, including for delivery of an agent to the corneal layer. Lamellae comprising collagen are present in corneal tissue. In some embodiments, the device shaft and tip of the shaft are configured to provide for selectively contacting lamellae in corneal tissue for delivery of an agent to the corneal tissue, such that the agent spreads along the lamellae, selectively within planes defined by the lamellae in the corneal tissue.

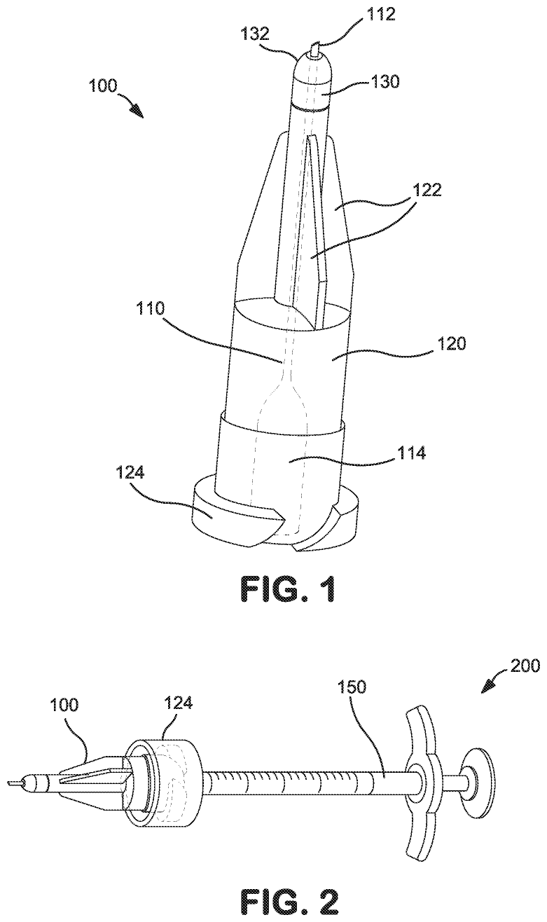

Referring to FIG. 1, an example embodiment 100 of a precise corneal injection (PCI) device is shown. PCI device 100 comprises a shaft 110, a hub 120, and a stop region 130.

Shaft 110 includes a needle tip 112 and in some embodiments a hollow pathway 114 for delivering a material to tip 112. Shaft 110 can be formed, for example, of metal or other suitable material using any method known in the art. For example, the device shaft can comprise a stainless steel, including but not limited to alloy 304 or 310. Hollow pathway 114 can be in any shape suitable for delivering the selected injectable material. In the embodiment of FIG. 1, tip 112 is a microneedle with a bevel 116. The dimensions of tip 112 can vary depending on the injectable material. As used herein, the term “PCI needle length”, “needle tip length”, and “tip length” are used interchangeably. In some embodiments, the length of the needle refers to the portion of the device that extends beyond the stop region.

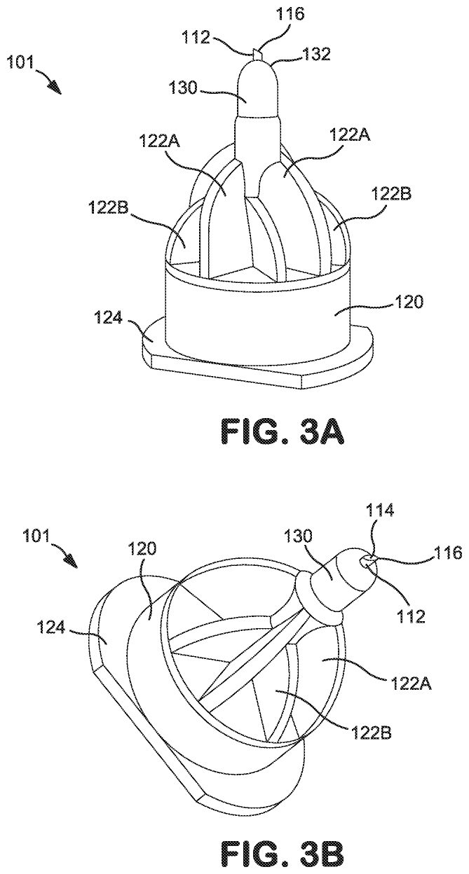

Hub 120 provides a housing for shaft 110 as well as optional features for connecting PCI device 100 to other devices. For example, in the embodiment of FIG. 1, hub 120 includes ribs 122, which can provide mechanical strength and gripping surfaces for PCI device 100. In this embodiment there are four ribs 122. Hub 120 is also shown with connector 124 on the proximal end of PCI device 100. Connector 124 can be of any shape suitable for connecting to the selected therapeutic device. For example, if PCI device 100 is to be attached to a syringe (see, e.g., FIG. 2), connector 124 can be in the form of a Luer connector. However, it will be appreciated by those of skill in the art that other connector configurations are possible. Hub 120 can comprise a polymer material.

Stop region 130 is disposed near the distal end of hub 120. Stop region 130 assists in controlling the depth of penetration of tip 112 and therefore generally has a larger diameter than shaft 110. In particular, stop 130 provides a precise length for tip 112, which can be configured for a desired treatment method. Additionally, stop region 130 has a rounded or beveled stop end 132. Stop 130 can be formed either separately from or integrally with hub 120 and can include a biocompatible elastomeric material such as silicone. It is further possible to form stop region 130 in multiple steps. In some embodiments, a base layer of stop region 130 can be formed integrally with hub 120 and then subsequently coated with an elastomeric material.

Referring now to FIG. 2, a PCI system 200 is illustrated. PCI device 100 is connected with a syringe 150 via connector 124. Syringe 150 can be of any suitable type known in the art. Syringe selection can depend, for example, on the material type and quantity to be injected into the cornea. PCI device 100 can be configured for both standard and low dead space syringes. Also, PCI device 100 can itself be configured such that hollow pathway 114, which is in communication with the delivery pathway of syringe 150, has configurable volume. The dimensions of hollow pathway 114 can be configured to include standard volume, low dead space, or other volumetric dimensions. This advantageously allows both small volumes and precise quantities of injectable material to be administered. Precision can be further improved through metered syringe pumps comprising reservoirs. Other configurations will further be apparent to one of ordinary skill in the art upon review of the instant disclosure. Although PCI device 100 is assembled to syringe 150 by connector 124 in the embodiment of FIG. 2, it is also conceivable that PCI device 100 can be formed integrally with a syringe or other treatment device.

Different parameters can be employed in the preparation of a device in accordance with the presently disclosed subject matter, including but not limited to length/diameter/size of opening of shaft 110 and/or tip 112, length and angle of bevel 116, volume for injection, and the like. Also, sizes and configurations of hub 120, shaft 110, and tip 112 can vary based on the species of the subject (e.g., human, equine, canine, ovine, etc.); based on the intended therapeutic; and/or based on the intended tissue target. For example, the target region of the eye can have a thickness and the dimension of tip 112 and/or bevel 116 can comprises a length that is less than the thickness of the target region of the eye.

By way of additional example and not limitation, the length of tip 112 can range from 0.1 to 1 mm, including 0.1, 0.2, 0.3, 0.4, 0.5, 0.6, 0.7, 0.8, 0.9, and 1.0 mm. Tip length can thus be less than corneal thickness. The length of bevel 116 can be less than the tip length. For example, the length of bevel 116 can range from 0.1 to 0.8 mm, including 0.1, 0.2, 0.3, 0.4, 0.5, 0.6, 0.7, and 0.8 mm. Exemplary shaft and/or tip gauge can range from about 30 to about 40 gauge (G), including 31G, 32G, 33G, 34G, 35G, 36G, 37G, 38G, 39G. and 40G.

A representative, non-limiting shape of stop end 132 is bullnose or conical. By way of example and not limitation, a bullnose stop end 132 can have a radius of curvature ranging from about 0.2 mm to about 1.5 mm, including 0.2, 0.3, 0.4, 0.5, 0.6, 0.7, 0.8, 0.9, 1.0 mm, 1.1 mm, 1.2 mm, 1.3 mm, 1.4 mm, and 1.5 mm. A conical-shaped stop end 132 can have a cone angle ranging from about 45 degrees to about 150 degrees, including 45, 50, 55, 60, 65, 70, 75, 80, 85, 90, 95, 100, 105, 110, 115, 120, 125, 130, 135, 140, 145, and 150 degrees.

Method of preparing a device of the presently disclosed subject matter are provided. For example, in configuring shaft 110 and tip 112, lasers can be employed to cut the shaft and tip to appropriate dimensions, shapes, and the like to provide for selective contacting of a predetermined target region of the eye. In some embodiments, the laser machining is used in conjunction with a standard imaging device, such as optical coherence tomography or high frequency ultrasound. By way of example and not limitation, a 50-70 megahertz (MHz) ultrasound can be employed. The imaging technique determines the thickness and/or depth of the target region of the eye to be contacted. In some embodiments, the information is used in the laser machining of a desired configuration of device to reach the target region of the eye to be contacted, such as a lesion to be treated.

Hub 120 can be prepared using a three-dimensional (3D) printing technique. In some embodiments, the 3D printing technique is controlled to provide a desired configuration based on the target region of the eye to be contacted. In some embodiments, the information obtained through the imaging technique that is determines the thickness and/or depth of the target region of the eye to be contacted is used in the 3D printing of the hub. Materials that can be employed in making hub 120 are including but not limited to polymer materials, including but not limited to biocompatible polymer materials, including but not limited to polypropylene (PP), polyethylene (PE), polyether ether ketone (PEEK), acrylonitrile butadiene styrene (ABS), a silicone, and the like. In some embodiments, hub 120 comprises a surface. In some embodiments, the surface comprises or is coated by biocompatible elastomer, optionally wherein the biocompatible polymer comprises a silicone.

In some embodiments, stop region 130 can comprise a biocompatible elastomeric material, such as but not limited to a silicone. Stop region 130 can also be prepared using a three-dimensional (3D) printing technique. In some embodiments, the 3D printing technique is controlled to provide a desired configuration based on the target region of the eye to be contacted. In some embodiments, the information obtained through the imaging technique that determines the thickness and/or depth of the target region of the eye to be contacted is used in the 3D printing of stop region 130. Stop region can 130 also facilitate the selective contacting of a target region of the eye. In some embodiments, the tip lengths as described herein are employed, such as but not limited to 610 μm and 700 μm, are employed, such that only this length extends beyond the stop region. PCI device 100 is configured so that only this length extends beyond the stop region.

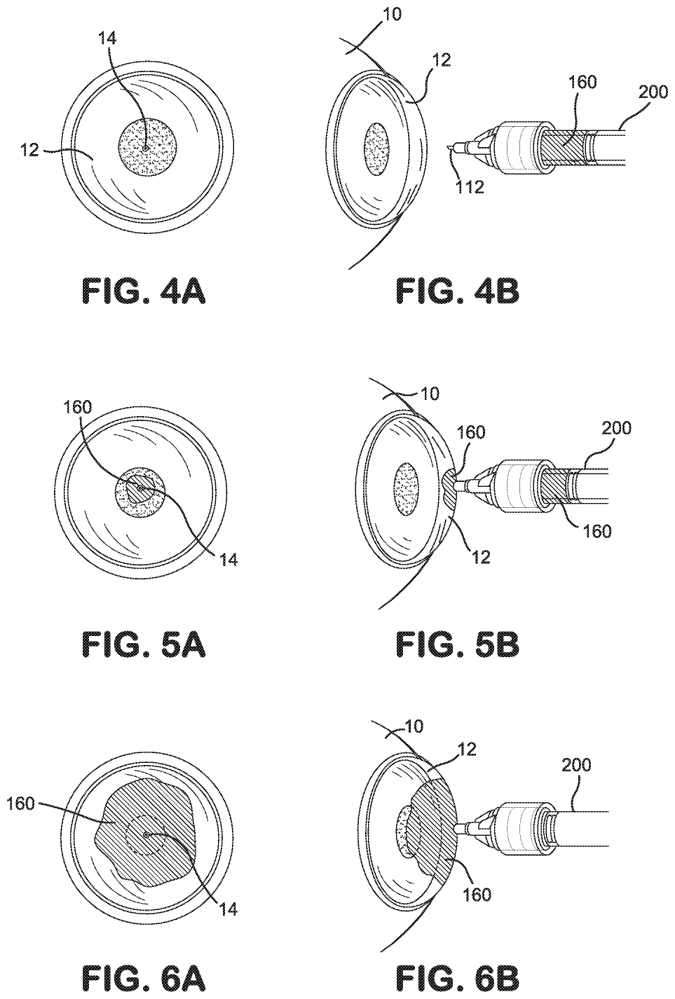

FIGS. 3A-3B illustrate two views of another example embodiment of a PCI device, generally designated 101. PCI device 101 has features similar to those of PCI device 100, including hub 120, stop region 130, and needle tip 112. PCI device 101 is connected to syringe 150 by connector 124. In the embodiment of FIGS. 3A-3B, hub 120 has eight ribs: four tall ribs 122A and four short ribs 122B. Other configurations of ribs are also possible. It is further possible for hub 120 to include additional ergonomic features such as longitudinal ridges, knurling, etc. (not shown).

In some embodiments, the presently disclosed device provides precise delivery of therapeutic drugs and gene therapy virus to the corneal stroma. The presently disclosed device also delivers a therapeutic to a specific location in the cornea (e.g., within a corneal stromal infection). Aspects of the presently disclosed device include ease of use, precise treatment, and safety of the device (e.g., mitigated risk of perforating the eye, which is common with standard injection techniques).

In some embodiments, the presently disclosed device is configured for implemented by placing it perpendicular to the target region of the eye, such as the cornea. However, the implementation can varied as might be appropriate depending on the region of the eye to be target, as would be apparent to one of ordinary skill in the art upon a review of the instant disclosure. Further, the configuration of the device is adapted as needed based on the implementation.

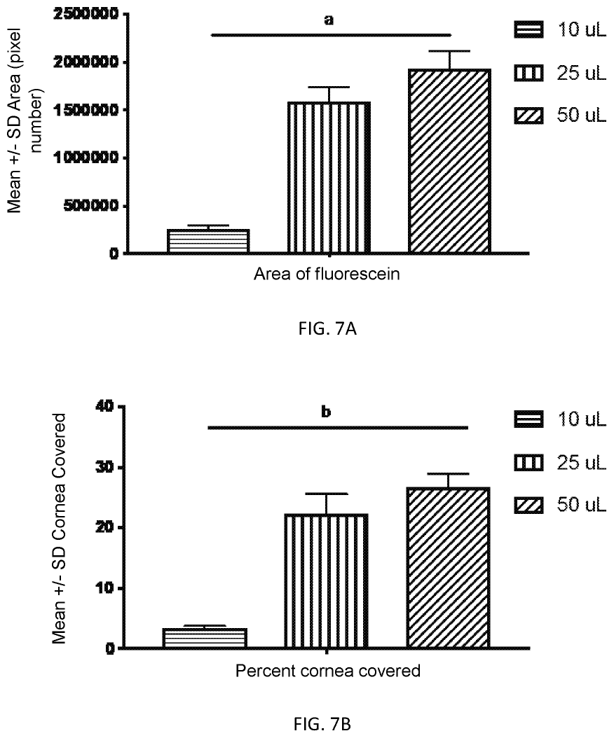

Referring now to FIGS. 4A-4B through 6A-6B, an example method of therapeutic use of PCI system 200 is illustrated. PCI system 200 can advantageously be performed on patients without use of magnification and with only local anesthesia. FIGS. 4A-4B show a PCI system 200 containing an amount of injectable material 160. PCI system 200 is positioned near eye 10 using any appropriate positioning method, in preparation for treating the cornea 12. An injection site 14 is selected. Injection site 14 can be, for example, a center of cornea 14. Tip 112 is then inserted into cornea 12. The presence of stop region 130 not only controls the depth of needle insertion but can also assist in reducing or preventing this possible damage, by providing a smooth surface to slow or stop corneal motion. Configuration of the bevel and injection tip of the needle is optimized for the tissue types, such as cornea or sclera, to facilitate injection in these tissues.

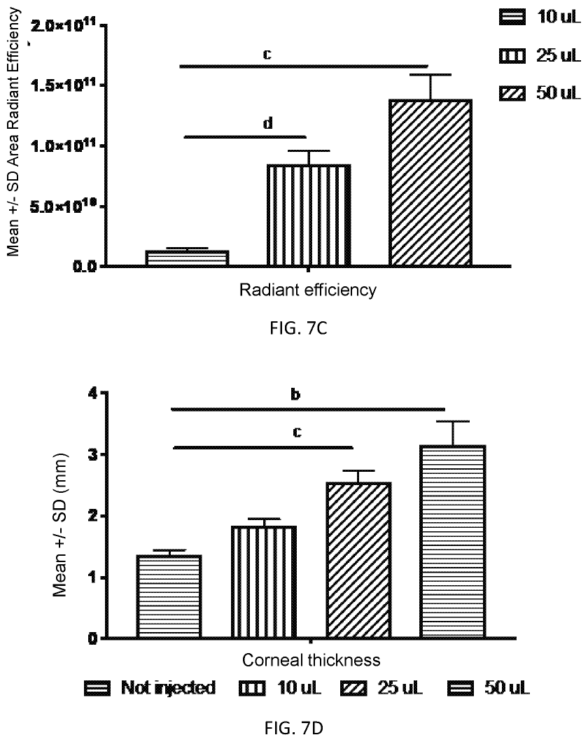

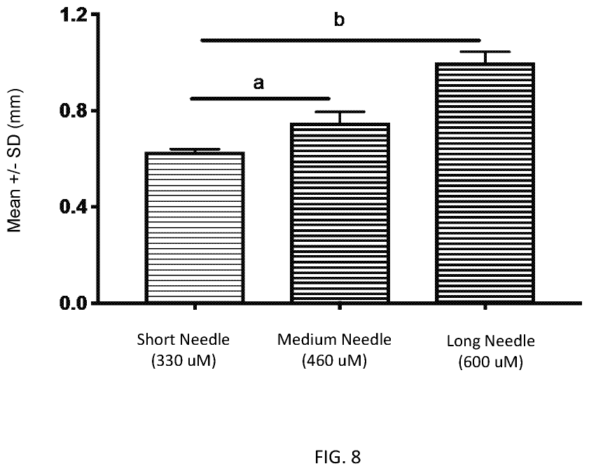

FIGS. 5A-5B show PCI system 200 inserted in cornea 12 at the predetermined depth of injection. As described hereinabove, this depth can be configured to deliver therapy to any corneal layer. Injectable material 160 is inserted into cornea 12, spreading outwardly from injection site 14. In FIGS. 6A-6B injectable material 160 fully administered to cornea 12. As can be seen in these Figures, the amount of injectable material is selected so that the material remains only in the cornea.

Any suitable therapeutic can be administered, e.g. injected, in accordance with the presently disclosed subject matter. By way of example and not limitation, the presently disclosed device can be used to deliver any currently applied topical ocular medications, such as steroids, antibiotics. NSAIDS, anti-glaucoma products, and the like. Furthermore, gene therapy (including any suitable vector) and stem cell therapy, viruses (including but not limited to adeno-associated viruses (AAV)), and viscoelastics (or other surgical enhancements for corneal or lasik surgeries) can also be delivered by the presently disclosed device.

Any corneal disease can be treated, including infectious, immune-mediated, genetic (e.g., MPS-1), neovascular, or degenerative diseases. Furthermore, the presently disclosed device can improve drug penetration or directly provide therapy for ocular anterior segment diseases, such as uveitis, glaucoma, trauma, etc. The device can also be used to facilitate surgery, such as separating Decemet's membrane from the stroma for deep lamellar keratoplasty.

Thus, representative corneal diseases include but are not limited to keratitis—Inflammation of the cornea that is commonly caused by infections, but can also be caused by include improper use of contact lenses, autoimmune disease, and injury; corneal dystrophy, in which parts of the cornea become cloudy due to buildup of cellular material; Stephens-Johnson Syndrome, in which painful blisters form on mucous membranes that are a result of allergic reactions to drug or from a viral infection; mucopolysaccharidosis type 1 (MPS1)-associated blindness (or other MPS diseases), which is a genetic disease wherein glycosaminoglycans accumulate, resulting in corneal clouding; RPE65 mutation-associated retinal dystrophy, which is a genetic disease characterized by absent RPE65 activity, resulting in impaired vision.

Nearly anything that can be a fluid or fine power can be administered, injected or otherwise delivered to the target region of the eye, including uses such as improvement for imaging (e.g. gadolinium for MRI), inks for cosmetic or therapeutic tattooing, or for precise placement of micro-electronics (e.g., visual aids, intraocular pressure gauges, and the like).

The quantity of agent applied can depend on the condition requiring application of the agent, the tissue being contacted and/or species to which it is applied. Representative ranges of volumes of agent include 1 to 200 μL of fluid volume, including 1, 5, 10, 20, 30, 40, 50, 60, 70, 80, 90, 100, 10, 20, 30, 40, 50, 60, 70, 80, 90, and 200 μL. Agents to be applied are formulated at standard concentrations as would be known and apparent to one of ordinary skill in the art upon a review of the instant disclosure.

A kit of parts for selectively contacting a target region of the eye is also provided. In some embodiments, the kit of parts can comprise one or more of the presently disclosed device and a container for the one or more device. Each device in the kit can comprise a different configuration, such a configuration for a range of different tissue types; a range of different species of subject; a range of gauges; and/or a range of shaft, tip, and/or bevel lengths, agents, and/or openings. In some embodiments, each device is a single use device.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

Porcine cadaver corneas were injected axially with increasing volumes of fluorescein using a purpose-designed, fixed-depth, precise corneal injection (PCI) needle. Color images, high frequency ultrasound (HFU), and fluorescent imaging were obtained post-injection to evaluate corneal thickness (CT), fluorescein distribution, and intensity. Additionally, eyes were injected using PCI needles of various tip lengths to determine depths of injection by HFU. Finally, corneas were injected with an AAV8 reporter at a fixed dose in escalating volumes. GFP fluorescence was evaluated by live imaging and histology while vector genomes and derived cDNA were quantitated by PCR.

After injection with the PCI needle, CT increased significantly with a direct correlation of volume to area infiltrated (p<0.0001). Depth of injection was consistent and correlated to needle tip length. The fixed dose of AAV-GFP in the lowest volume resulted in earlier GFP fluorescence in a smaller area while the higher volume demonstrated later-onset GFP over a broader corneal area and significantly increased genome abundance.

The purpose of this Example was to determine reproducibility, depth, and extent of corneal distribution ex vivo following stromal injection of fluorescein or a gene therapy vector, adeno-associated virus (AAV) harboring green fluorescent protein (GFP) cDNA, using a purpose designed precise corneal injection (PCI) needle in accordance with the presently disclosed subject matter. The data demonstrate that PCI needles provides reproducible, uniform, and precise site-specific corneal drug delivery by a simple macroscopic procedure. PCI needles allowed injection consistency sufficient to elucidate volume dependent AAV vector distribution, genome persistence, and overall transduction efficiency in porcine corneas ex vivo. When coupled with its ease of use, PCI needles reduce consequential variables, compared to current corneal injection technologies, and are well-suited for drug applications for corneal disease including gene therapy.

### Methods

Ex vivo porcine cadaver tissues were used in all sections of this study; no live animals were used. Ocular tissues were placed on ice immediately after removal at a local USDA-certified slaughter house, transported to the research facility on ice, and used for experiments within 8 hours after animal death. Only eyes with normal, clear corneas based on visual inspection were used. Injections were made on whole porcine cadaver eyes placed in a fixation device (Mastel Mandell Eye Mount, Mastel Precision Surgical Instruments, Rapid City, S. Dak., USA) with the vacuum adjusted to provide a normotensive intraocular pressure of 15-20 mmHg as measured by a TONOVET™ tonometer (Icare, Finland). For AAV-GFP injections, the eyes were first irrigated with 1% betadine solution and sterile saline, then the corneas were excised and, for injections, were placed into a sterile artificial anterior chamber device (Barron Artificial Chamber, Katena Products, Inc., Denville, N.J., USA) inflated with sterile balanced salt solution (BSS, Alcon Laboratories, Fort Worth, Tex., USA) to create an anterior chamber pressure of 15-20 mmHg. Following injections, corneas were then cultured.

### Corneal Injection and Distribution of Fluorescein

Corneal injections were made with either a 31-gauge insulin syringe (BD Ultra-Fine™ 8 mm 31G syringe, Becton, Dickinson and Company, Franklin Lakes, N.J., USA) or a purpose designed precise corneal injection (PCI) needle in accordance with the presently disclosed subject matter; a 34-gauge needle with a defined, fixed depth, and bevel configuration optimized for corneal intrastromal injections.

Intrastromal injections were made with the 31-gauge insulin syringe (BD Ultra-Fine™ II Short Needle Insulin Syringe 31G lec 5/16″, Becton, Dickinson and Company, Franklin Lakes, N.J. USA) as previously described [1]. The needle was directed obliquely and horizontally from the temporal limbus and extended to the central cornea with the bevel pointed down followed by slow injection of 50 μL of 0.01% sodium fluorescein (AK-Fluor® fluorescein injection, USP, Lake Forest, Ill.) in BSS. These injections were repeated in a total of 4 eyes and then imaged.

In a separate set of eyes, PCI needles were used to inject directly into the axial corneal stroma from an anterior, perpendicular approach. For these injections, a 650 μm length PCI needle was used to inject 10 μL, 25 μL, or 50 μL of 0.01% fluorescein into the central cornea. For the 10 μL injections, a 50 μL glass syringe (Microliter 700 Series Syringe, Hamilton Company, Reno, Nev., USA) was used. For the 25 and 50 μL injections, a 0.25 mL Sword Handle Fixed Male Medallion syringe was used (Merit Medical, Inc., South Jordan, Utah, USA). Injections were performed in triplicate, with theme additional eyes serving as un-injected controls.

Images of all corneas were collected immediately after injection (time 0) and repeated 3 and 24 hours later using digital ocular photography (Nikon D200, AF-S DX Micro NIKKOR 85 mm f/3.5G Lens, Nikon Corporation, Tokyo, Japan) with fixed magnification. Digital images were analyzed to determine distribution of fluorescein by measuring area (pixel counts) of the visible fluorescein using ImageJ image processing (ImageJ 1.51a, National Institutes of Health, Bethesda, Md., USA). Eyes were maintained at room temperature in a humidified plastic container for 24 hours after injection. Percentage of total corneal coverage of the injection was measured by outlining the corneal limbus and collecting total corneal pixel counts by ImageJ, then the following equation was used: fluorescein area (pixel count)/total corneal area (pixel count)×100=percent of corneal fluorescein coverage.

To evaluate the location and depth of injection and resulting corneal thickness, high frequency ultrasound (HFU) was performed with a 50 MHz linear probe in B mode (Aviso™, Quantel Medical, Bozeman, Mont., USA). Sagittal images were obtained of the central cornea prior to injection, immediately following injection, then at 3 hours and 24 hours after injection. Corneal thickness was measured using the ultrasound instrument caliper function by measuring the central portion of each image from the surface epithelium to endothelium. To evaluate fluorescence intensities and area, radiant efficiency (RE) ([(photons/s)/(μW/cm²)]) was calculated for each eye at 1, 3, and 24 hours after injection using the IVIS® Spectrum imager (Caliper Life Sciences, Hopkinton, Mass., USA) and Living Image® software using the following parameters: 1 second exposure, F stop 4, medium binning, and GFP excitation filter. Using the IVIS imager, region of interest (ROT) was gated for each cornea and the RE (intensity of fluorescence) was automatically calculated. RE values were adjusted for background fluorescence by subtracting the average RE of control corneas at the given time point, as previously described [2,3].

### Evaluation of Depth of Injection and Needle Penetration

To determine the depth of injection using PCI needles in accordance with the presently disclosed subject matter, a separate set of eyes (n=3/group) were injected using either a 330 μm (short), a 460 μm (medium), or a 600 μm (long) PCI needle. A 50 μL glass syringe was used to inject each eye with 10 μL of 0.01% sodium fluorescein. Digital photography and HFU images were obtained for each eye prior to, and immediately after, injection. For comparison and assessment of the injection location, on the ultrasound image the distance from the corneal epithelium to the center of the injection site was measured using the ultrasound calipers for each eye.

To determine the extent of the PCI needle penetration into the cornea as a function of needle tip length, porcine cadaver eyes were fixed in a Mastel corneal vacuum mount, and either a 600 or 700 μm tip length PCI needle was inserted into the cornea, but without fluid injection. Following removal of the PCI needle, imaging of the corneal epithelium to endothelium was done consecutively using confocal microscopy (Heidelberg Retina Tomograph 3 with Rostock Corneal Module, Heidelberg Engineering, GmBH, Dossenheim, Germany). Confocal imaging was also performed on a non-injected normal cornea.

Transgene Fluorescence after Localized Corneal Stromal Injection of AAV-GFP

To demonstrate feasibility of delivering gene therapy to the cornea using the PCI needle, intrastromal injection of self-complementary (sc) [4,5], AAV8-EF1α-GFP was performed. Self-complementary AAV-GFP vectors, provided by the Vector Core at University of North Carolina, Chapel Hill, N.C., USA, were used in this study and production and characterization of AAV vectors were done as previously described 16,71. In a separate set of eyes, following irrigation with 1% betadine and BSS, corneas were excised and fixated in a sterile artificial anterior chamber (Barron Artificial Chamber, Katena Products, Inc., Denville, N.J., USA). A fixed dose of scAAV8-EF1α-GFP (1.0×10¹⁰ viral genomes [vg]) was diluted in 10, 25, or 50 μL of sterile saline and injected intrastromally using a PCI needle. Injections of each volume of virus or a BSS control was performed in triplicate, then the corneas were removed from the artificial anterior chamber, and placed into 6 well-culture plates with Dulbecco's Modified Eagle's Medium (DMEM) with 10% fetal bovine serum and 1% penicillin-streptomycin (Sigma-Aldrich, St. Louis, Mo., USA). Culture plates were incubated at 37° C. and 5% CO₂. Corneas were washed in sterile PBS and medium was changed daily. Corneas were imaged for GFP fluorescence using the Spectrum IVIS imager (Caliper Life Sciences, Hopkinton, Mass., USA) daily post-injection as described previously. GFP intensities were identified, a ROI was gated, and RE ([(photons/s)/(μW/cm²)]) was calculated daily for each cornea. Following imaging on day 7, corneas were sectioned and one half of the cornea was fixed in 10% buffered formalin for 24 hours, processed, embedded and sectioned for GFP immunofluorescence detection, while the other half was frozen on dry ice and stored at −80° C. for quantitative analysis of transgene expression via RT-PCR.

### Corneal GFP Immunofluorescence

Corneas were excised, fixed, embedded in paraffin, and sectioned at a thickness of 5 μm. Immunofluorescence was performed as previously described for ocular tissues [6,8]. Sections were deparaffinized by incubating slides twice in xylene for 10 min each, followed by immersing slides sequentially in two rounds of 100% (3 min each), 95% (1 min), and 80% (1 min) ethanol solutions, and then in distilled water for 5 min. Antigen retrieval was performed by heating the slides to 98° C. in citrate-based (pH 6.0) antigen unmasking solution (Vector Laboratories, Burlingame, Calif., USA) before staining. Non-specific staining was blocked by using PBS containing 10% normal goat serum, 0.025% Triton X-100 plus 1% bovine serum albumin (BSA) prior to overnight incubation with the primary antibody. The GFP primary antibody (1:500) (AVES Labs, Inc., Tigard, Oreg., USA) and goat anti-chicken secondary antibody (Alexa Fluor® 488, 1:1000) (Abcam, Cambridge, Mass., USA) were used for GFP expression. After the staining, slides were mounted and counter stained with ProLong™ Diamond Antifade Mountant with DAPI (ThermoFisher Scientific, Waltham, Mass., USA) [6].

Quantification of Corneal GFP Transgene Expression by qRT-PCR

Corneal GFP transgene expression following intrastromal injection was quantitatively analyzed by qRT-PCR as previously described for ocular tissues using probe detection [6]. Isolated total RNA was subjected to DNase I treatment (Ambion® by ThermoFisher Scientific, Waltham, Mass., USA) before reverse transcription was performed. cDNA was then synthesized with the Second Strand Synthesis Kit (Invitrogen, Carlsbad, Calif., USA) in the presence and absence of reverse transcriptase (RT). qPCR of recovered porcine β-actin cDNA was performed using SYBR Green detection with the forward primer: CTGCGTCTGGACCTGGCTG (SEQ ID NO:1), and the reverse primer: ACGCGGCAGTGGCCATCTC (SEQ ID NO:2). The amplified products were validated by a melting curve analysis to assure specific amplification. qPCR amplification of GFP transgene was performed with GFP primers (Forward primer 5′-ccatgccgagagtgatcc-3′(SEQ ID NO:3); reverse primer 5′-gaagcgcgatcacatggt-3′(SEQ ID NO:4)) and the Universal probe #67 (Roche, cat. no. 04688660001). When the result was below the lowest detection limit, the results were considered as negative. qPCR reactions in this study were performed with a Roche 480 Lightcycler. The GFP expression was normalized to that of β-actin. The results are presented as the relative fold change calculated using 2-ΔΔCt method.

Viral Genome Detection by qPCR.

To detect the viral genome (vg) in the corneas, gDNA from corneas were isolated using DNeasy Blood and Tissue Kit (Qiagen, Valencia, Calif., USA). Vector genome was quantitatively analyzed by qPCR utilizing the probe technology as described above. In short, the amount of vector-specific GFP genome copies was standardized against an amplicon from a single copy housekeeping gene β-actin. qPCR was carried out with an initial denaturation step at 95° C. for 10 min, followed by 45 cycles of denaturation at 95° C. for 10 s, and annealing/extension at 56° C. for 45 s for the GFP probe detection. The results are presented as the relative fold change calculated using 2-ΔΔCt method.

### Statistical Analysis

Associations among fluorescein area, corneal thickness, and radiant efficiencies were determined using ANOVA, student t test, and Tukey's post hoc analysis for multiple comparisons. Differences were considered significant at p≤0.05 and all probabilities and results were calculated using computerized statistical software (JMP® Pro, v. 13.2; SAS Inc., Cary, N.C., USA).

### Results

### Corneal Injection and Distribution of Fluorescein

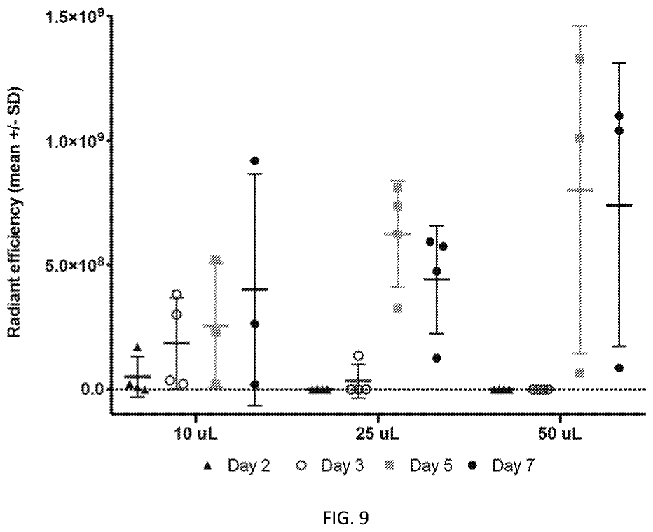

Referring to FIGS. 7A-7D, corneal intrastromal injections of fresh porcine whole globes with 50 μL of 0.01% sodium fluorescein were performed with either a standard, 31-gauge needle or a PCI needle. The corneal intrastromal injections using the standard needle resulted in variable location (i.e., not consistently axial), inconsistent corneal distribution, and different stromal depths. Additionally, the 31-gauge standard needle resulted in a 25% failure rate defined by endothelial perforation with injection into the anterior chamber. In contrast, intrastromal injection using the 650 μm PCI needle into ˜1000 μm thick cadaver porcine cornea, the fluorescein injection radiated concentrically from the injection site in an even manner without leakage from the injection site and no intracameral penetration. Not only was the injection using the PCI needle precisely and repeatedly placed in the central stroma (without any off-target injection), there was a direct injection volume/area correlation that significantly increased with volume (p<0.0001), as measured by the fluorescein area, intensity, and percent of corneal coverage (see, e.g., FIGS. 7A, 7B). Consistently, HFU investigation of central corneal thickness demonstrated a direct correlation between corneal distension and injection volume (p<0.04) (FIG. 7D). Twenty-four hours after injection, as seen in both color and fluorescent imaging, the corneal fluorescein deposit diffused through the corneal stroma to involve 40% of the cornea with the 10 μL injection to 80% with the 50 μL injection. Concomitantly, the corneal thickness returned to baseline by 24 hours.

### Evaluation of Depth of Injection and Needle Penetration

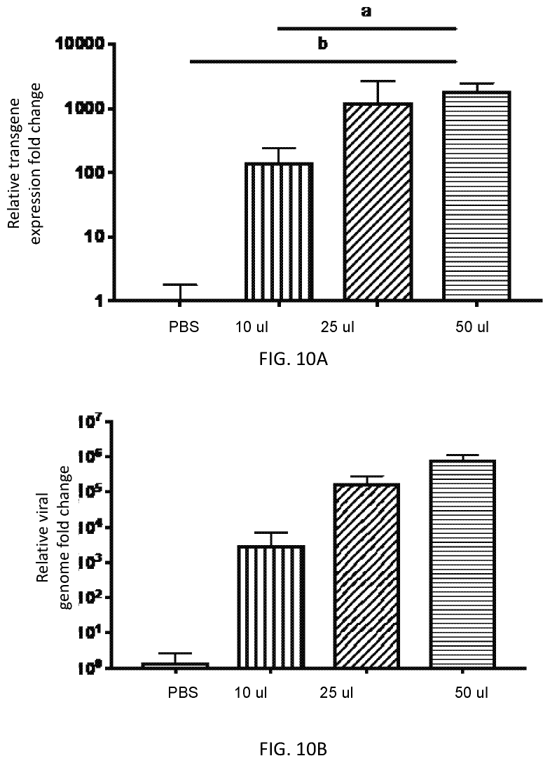

Referring to FIG. 8, evaluation of HFU images immediately following a 10 μL injection of 0.01% fluorescein in ex vivo porcine corneas demonstrated the depth of injections was significantly (p<0.004) and directly related to the length of the needle tip (330-600 μM). Confocal microscopy showed that the PCI needle created a linear incision into the corneal epithelium with minimal changes to the surrounding tissue. The depth of penetration of a 600 μM and 700 μM PCI needle was 604 and 689 μM, respectively. Slit-like incisions were visible through the stromal layers. At the edge of the incisions and at the maximum depth of the needle penetration there was a more dense, brighter appearance of the corneal lamellae, suggesting a slight compression of the tissue.

### Volume Enhances AAV Vector Transduction Efficiency and Distribution

Studies have suggested that AAV vectors may serve as therapeutics for corneal associated vision loss following intrastromal injection. In these cases, vector administration relied on use of obliquely oriented needles (27-31 gauge) using varying doses and administration volumes. Regarding transduction efficiency, reports in other tissues have demonstrated that administered volume influences AAV gene delivery. However, in the cornea inconsistent vector administration presents variables that confuse result interpretation. Therefore, the PCI needle was employed to standardize intrastromal injection variables for administration of a fixed dose of scAAV8-EF1α-GFP (1e10vg) in increasing volumes (10 μl, 25 μl, and 50 μl).

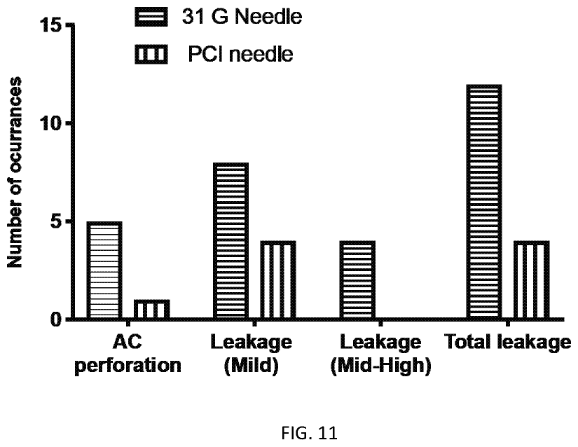

Referring to FIG. 9, GFP fluorescence was quantitated at days 2, 3, 5, and 7 post-injection by live imaging (Caliper Life Sciences, Hopkington, Mass., USA). The fixed vector dose administered in the lowest volume trended towards earlier GFP detection at 48 h and 72 hr post-injection. At later time points, vector administered in the higher volumes demonstrated increased fluorescence compared to 10 μl. On Day 7 post-injection, total DNA and RNA was recovered from the corneas and analyzed by qPCR.

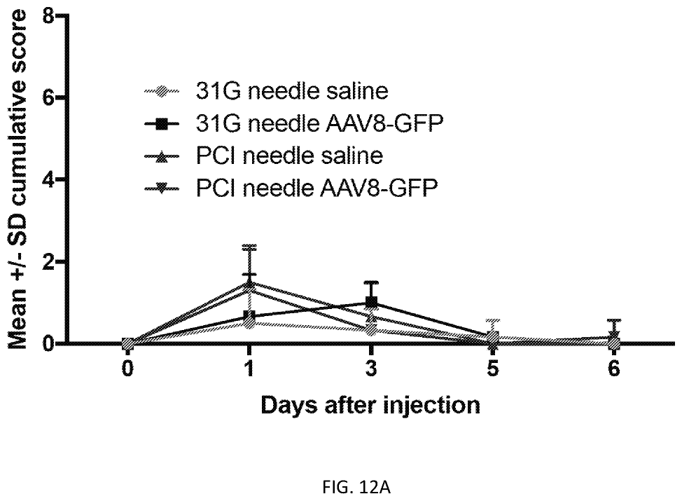

As shown in FIGS. 10A-10B, detection of vector genomes demonstrated a large significant increase in persistence directly correlated to injection volume. For instance, an approximate 100-fold increase in genome abundance was discovered for fixed-dose vector administered in 10 μl compared to 50 μl (FIG. 10A). Consistently, transgene derived cDNA also increased >10-fold when vector was administered in higher volumes (FIG. 10B).

### Corneal Immunofluorescence for GFP

To determine the effect of volume on vector distribution in the cornea, GFP immunofluorescence was performed after intrastromal fixed dose scAAV8-EF1α-GFP injection using PCI needles. Seven days post-injection, the corneas were prepared for histological analysis using GFP staining. The results revealed greater vertical and lateral distribution of GFP abundance within the corneal stroma directly correlated to increasing administration volumes.

### Discussion

Corneal injection using PCI needles provided simple and consistent drug distribution in the cornea in a repeatable and user-independent manner. The PCI needle of the presently disclosed subject matter allowed recognition that a fixed dose of AAV vectors administered in a higher volume increased vector genome persistence and both intensity and distribution of transduction.

Corneal intrastromal injections are currently applied in clinical cases of infectious keratitis (i.e., fungal and bacterial infections) and corneal neovascularization [9-13]. Compared to topical drug delivery, intrastromal injection offers several advantages including localized intra-organ delivery, high local drug concentrations, and prolonged tissue exposure to the injected drug. Emerging drug contexts, such as AAV gene therapy also benefits from intrastromal administration thereby minimizing off target transduction and/or environmental shedding compared to topical or subconjunctival administration. However, conventional corneal intrastromal injections frequently require patient anesthetization as well as equipment such as surgical microscopes. Additionally, conventional 27-31 gauge needles result in variable injection depth, drug spread, and, depending on the species, are quite challenging with an endothelial perforation rate of approximately 25%. To overcome these hurdles associated with intrastromal injection, 34 gauge PCI needles were created with different tip lengths to allow versatility in a manner that circumvents the concerns associated with standard needles. Compared to a standard 31 gauge needle, PCI needles allowed repeatable, axial corneal intrastromal injection without leakage to the ocular surface or intracameral penetration. Furthermore, by selecting appropriate PCI needle lengths, injection to the superficial, midstromal, or deep stromal layers of the cornea was specifically targeted in porcine corneas. Confocal imaging demonstrated that the depth of PCI penetration into an ex vivo cornea approximates the length of the PCI needle. This allows the use of the PC needle to target specific corneal sites by selecting an appropriate length PCI needle to reach the diseased tissue location and depth, which is determined by the clinician following use of high resolution imaging, such as HFU or optical coherence tomography. Site specific targeting of disease can possibly reduce toxic side effects, immune complications, and systemic exposure of drugs including gene therapy vectors and their transgenes. Targeted treatment can also possibly reduce associated costs by reducing the required dose, the frequency of use, and secondary treatment of adverse events or side effects. These benefits of the use of the fixed depth PCI needle combined with the ability to use the device to make precise injections in a patient without magnification and with only local anesthesia suggest that the PCI needle could be used for many corneal therapeutics and easily in the field by first-responders and military personnel, as examples.

This Example demonstrated that a single injection intrastromally using the PCI needle can provide wide therapeutic exposure to the cornea, resulting in diffusion from a central injection area to involve nearly 50-80% of corneal surface over 24 hours. This centripetal spread likely occurred along corneal stromal lamellar planes and the diffusion resulted in a return of the corneal thickness to baseline within 24 hours of the injection. Although these were cadaver ex vivo eyes, the results were similar to previous reports in vivo in rabbits and canines, where the corneal opacity associated with intrastromal injection had returned to normal clarity and thickness also by 24 hours after injection [1]. See also Example 2 herein below. Finally, AAV gene delivery throughout the cornea using the PCI needle also was shown to be feasible and defined the effect of volume on fixed dose vector transduction. Larger injection volume increased vector genome persistence, overall transduction efficiency and distribution of the transgene product, consistent with AAV vector administered by other injection routes [14,15]. Given that lower vector doses are associated with reduced production costs, decreased immunogenicity, and better therapeutic outcomes, adjustment of the administered volume offers an avenue for >5-fold enhanced transduction without rational or combinatorial engineering of enhanced AAV capsids.

The PCI needle can facilitate the clinical use of direct corneal therapeutics, which have been described using standard needles in several disease contexts. Use of corneal intrastromal injection has been described in pre-clinical and clinical studies for the delivery of anti-neovascularization drugs, anti-fungal drugs, riboflavin (for corneal cross-linking), and gene therapy, for example. The PCI needle could be used in these applications with an improvement in ease of use and precision of delivery of the therapeutics and to minimize the risk of corneal perforation or endophthalmitis.

Additionally, acquired and inherited diseases that result in corneal opacity have their origin in stromal abnormalities. Therefore, direct stromal injection using the PCI needle can be a precise, relatively atraumatic, alternative for conventional corneal gene therapy to allow safer, consistent, and precise administration in a clinician-independent manner.

The results of this Example evaluating corneal intrastromal injection using the PCI needle in ex vivo porcine eyes demonstrate precise, repeatable, and minimally invasive injections into the cornea for applications such as corneal drug delivery and viral vector delivery. The ability to provide a high tissue concentration and long exposure time of the drug at a desired location in the cornea stroma, without the concern of off target leakage of drug or viral vector, makes the PCI needle a versatile method for corneal intrastromal injection.

### References for Example 1

1. Hirsch M L, Conatser L M, Smith S M, et al. AAV vector-meditated expression of HLA-G reduces injury-induced corneal vascularization, immune cell infiltration, and fibrosis. Sci Rep. 2017; 7(1):17840. doi:10.1038/s41598-017-18002-9
2. Schaefer E, Smith SM, Salmon J, et al. Evaluation of Intracameral Pentablock Copolymer Thermosensitive Gel for Sustained Drug Delivery to the Anterior Chamber of the Eye. J Ocul Pharmacol Ther. March 2017. doi:10.1089/jop.2016.0181
3. Schaefer E, Abbaraju S, Walsh M, et al. Sustained Release of Protein Therapeutics from Subcutaneous Thermosensitive Biocompatible and Biodegradable Pentablock Copolymers (PTS gels). J Drug Deliv. 2016; 2016:1-15. doi:10.1155/2016/2407459
4. Hirata R K, Russell D W. Design and Packaging of Adeno-Associated Virus Gene Targeting Vectors. J Virol. 2000. doi:10.1128/JVI.74.10.4612-4620.2000
5. McCarty D M, Monahan P E, Samulski R. Self-complementary recombinant adeno-associated virus (scAAV) vectors promote efficient transduction independently of DNA synthesis. Gene Ther. 2001. doi:10.1038/sj.gt.3301514
6. Song L, Llanga T, Conatser L M, Zaric V, Gilger B C, Hirsch M L. Serotype survey of AAV gene delivery via subconjunctival injection in mice. Gene Ther. 2018; 25(6):402-414. doi:10.1038/s41434-018-0035-6
7. Hirsch M L, Li C, Bellon I, et al. Oversized AAV transductifon is mediated via a DNA-PKcs-independent, Rad51C-dependent repair pathway. Mol Ther. 2013. doi:10.1038/mt.2013.184
8. Hirsch M L, Conatser L M, Smith S M, et al. AAV vector-meditated expression of HLA-G reduces injury-induced corneal vascularization, immune cell infiltration, and fibrosis. Sci Rep. 2017; 7(1):17840. doi:10.1038/s41598-017-18002-9
9. Prakash G, Sharma N, Goel M, Titiyal J S, Vajpayee R B. Evaluation of Intrastromal Injection of Voriconazole as a Therapeutic Adjunctive for the Management of Deep Recalcitrant Fungal Keratitis. Am J Ophthalmol. 2008; 146(1). doi:10.1016/j.ajo.2008.02.023
10. Hu J, Zhang J, Li Y, et al. A Combination of Intrastromal and Intracameral Injections of Amphotericin B in the Treatment of Severe Fungal Keratitis. J Ophthalmol. 2016; 2016:1-7. doi:10.1155/2016/3436415
11. Niki M, Eguchi H, Hayashi Y, Miyamoto T, Hotta F, Mitamura Y. Ineffectiveness of intrastromal voriconazole for filamentous fungal keratitis. Clin Ophthalmol. 2014. doi:10.2147/OPTH.S63516
12. Liang SYW, Lee G A. Intrastromal injection of antibiotic agent in the management of recalcitrant bacterial keratitis. J Cataract Refract Surg. 2011. doi:10.1016/j.jcrs.2011.03.005
13. Hashemian M N, Zare M A, Rahimi F, Mohammadpour M. Deep intrastromal bevacizumab injection for management of corneal stromal vascularization after deep anterior lamellar keratoplasty, a novel technique. Cornea. 2011. doi:10.1097/ICO.0b013e3181e291a6
14. Hennig A K, Ogilvie J M, Ohlemiller K K, Timmers A M, Hauswirth W W, Sands M S. AAV-mediated intravitreal gene therapy reduces lysosomal storage in the retinal pigmented epithelium and improves retinal function in adult MPS VII mice. Mol Ther. 2004. doi:10.1016/j.ymthe.2004.03.018
15. Aschauer D F, Kreuz S, Rumpel S. Analysis of Transduction Efficiency, Tropism and Axonal Transport of AAV Serotypes 1, 2, 5, 6, 8 and 9 in the Mouse Brain. PLoS One. 2013. doi:10.1371/journal.pone.0076310

### Example 2

Gene therapy targeting cornea stromal diseases has led to the need for precise drug delivery to the cornea. Small gauge needles (e.g., 31G) are available, but their use results in variability with common perforation resulting in differences in drug distribution and efficacy. To allow consistent and precise corneal stroma drug delivery, a purpose-designed injection (PCI) fixed depth needle in accordance with the presently disclosed subject matter was developed. This study was designed to compare drug distribution between the current standard 31G and PCI needle.

Corneal gene therapy has recently been described or advocated for a diverse set of corneal abnormalities, such as decrease fibrosis, endothelial dystrophy, corneal dystrophies, prevention of corneal transplant rejection, and storage disease. The corneal stroma itself, a regular array of collagen lamellae separated by glycosaminoglycans and keratinocytes, which unlike corneal epithelium, do not have a rapid turnover and remain in a relatively quiescent state unless injured. Therefore, once corneal stromal cells are transduced, gene expression is typically long term [1]. Several methods have been described to transduce corneal stromal cells for gene therapy. Described methods include applying viral vectors following the creation of a surgical flap to expose the corneal stroma [2,3], creating a stromal pocket to apply the vector using femtosecond laser [4], and topical application of the viral vector following corneal epithelial removal or dessication [5,6]. All of these procedures may induce additional inflammation or corneal adverse effects, which may become acerbated when treating corneal disease. Therefore, direct stromal injection using the PCI needle in accordance with the presently disclosed subject matter provides a precise, relatively atraumatic method for corneal gene therapy to help this mode of therapy advance to routine corneal use.

### Methods

Normal New Zealand White rabbits were used in this study. A 31G or 318 μm PCI needle was used for instrastromal injections in anesthetized rabbits under an operating microscope. With either needle, the right eye received 25 μL of AAV8-GFP (1e9 viral genomes [vg]) while the left eye received 25 μL saline (n=6 each injection). Prior to injection and on days 1-6, 9, 14, and 16, slit lamp biomicroscopy, pachymetry, and intraocular pressure were performed. Additionally, in vivo GFP expression using a scanning laser ophthalmoscope (SLO) was done on days 6, 9, and 16. On day 18 after injection, rabbits were euthanized and eyes collected, and analyzed histologically, for GFP expression by immunofluorescence (IF or probe-based quantitative qPCR analysis. Serum neutralizing antibody to vector capsid was analyzed, Additionally, peripheral viral genome biodistribution was assessed by qPCR in peripheral blood, liver, spleen, submandibular and mesenteric lymph node (LN), kidney, heart, and skeletal muscle.

### Results

### Comparison of Intrastromal Injection Using 31G Vs PCI Needle In Vivo.

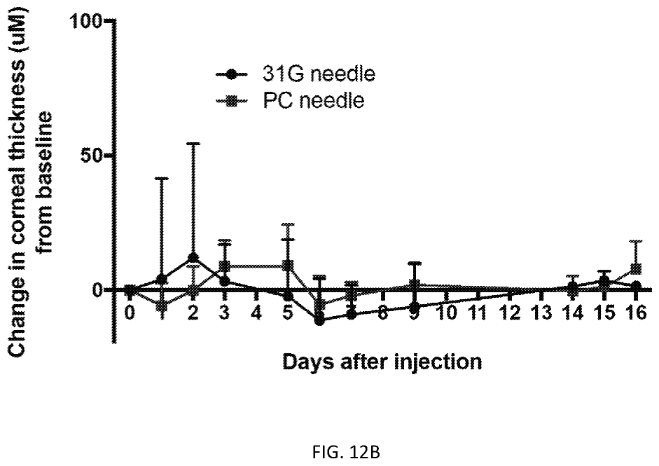

FIG. 11 is a comparison chart of 31G and PCI needle injections. Of 12 injections made with 31G needle, 5 resulted in anterior chamber (AC) perforations, 4 had moderate or high injection site drug leakage (8 with mild), but 10 achieved good intrastromal injections. In comparison using a PCI needle, only 1 injection was intracameral, 6 eyes had no leakage (2 moderate to severe), and 10 achieved good instrastromal injections. Using either the 31G or PCI needle, no adverse effects were observed and mean ocular inflammatory scores, IOP, and corneal thickness returned to near baseline by 24 hours and normalized in all eyes by 5 days after injection.

Intrastromal injection of 25 μL of BSS or AAV-GFP using either the 31G or PCI needle resulted in corneal opacity immediately after injection (A) which resolved Day 1 after injection. B. Following injection, anterior chamber perforation (5 versus 1), and injection site leakage (12 vs 6) were more frequent using the 31G vs the PCI needle. However, good stromal injections were achieved in 10/12 corneas with each needle.

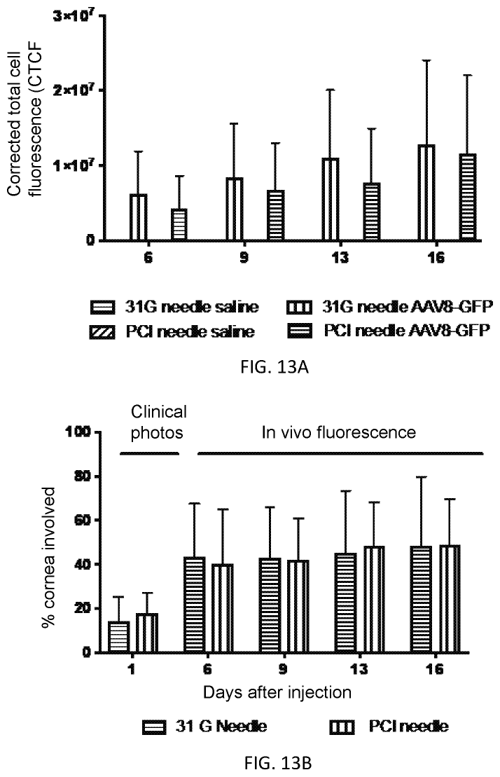

FIGS. 12A-12B compare ocular inflammation using 31G and PCI needles. Mean cumulative ocular inflammatory scores (FIG. 12A) and mean corneal thickness (FIG. 12B) following intrastromal injection using the 31G or PCI needle were measured over time. There were no significant differences in either mean inflammation or corneal thickness in eyes between needle types.

### In Vivo Expression of GFP

Although the area of corneal GFP fluorescence was not significantly different in eyes injected with 31G or PCI needles, the percentage area of the corneal infiltrated using the 31G and PCI needles was 14.3 and 18.2% immediately after injection, respectively. However, by 16 days after injection, the percentage area of corneal GFP expression was nearly 50%.

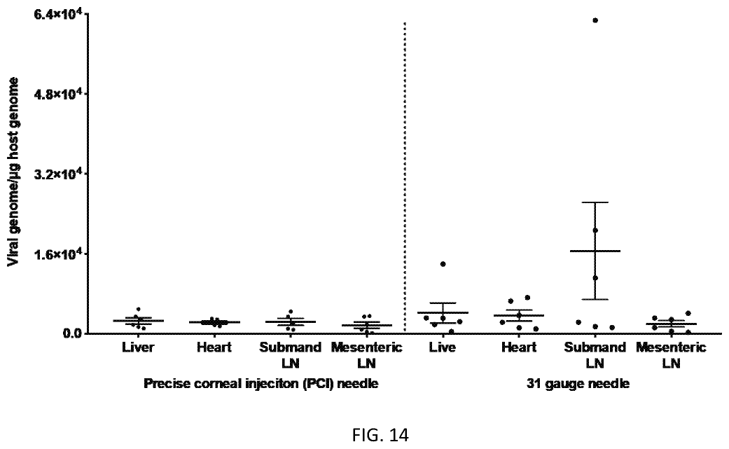

Referring to FIGS. 13A-13B, in vivo expression of GFP using a scanning laser ophthalmoscope (SLO) from days 6, 9, 13 and 16 after intrastromal injection of BSS (OS) or AAV8-GFP (OD) using either a 31G or PCI needle was measured. Fluorescence was not visible in the left eye, however, corneal expression was noted in increasing density in right eyes. Mean fluorescence using the 31G or PCI needle in right corneas increased at each time point after injection, but were not significantly different at any day (FIG. 13A). The area of fluorescence was higher at 6, 9, 13, and 16 days compared to the visible injection site immediately after injection, suggesting that diffusion of the virus beyond the injection site occurred (FIG. 13B).

### Viral Genome (VG) Distribution

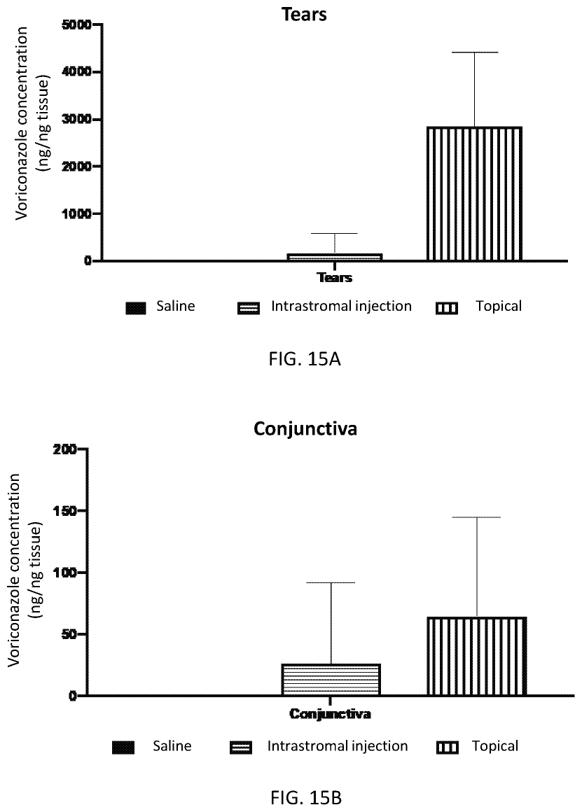

Viral genome copies were higher in peripheral tissues in animals injected with the 31G compared to the PCI needle, especially in the submandibular LN. As shown in FIG. 14, viral genome (VG) distribution following intrastromal injection of AAV8-GFP (1×10⁹ vg) using either 31G or PCI needle was measured. There was more variance among individual samples in all 31G groups, however, no significant difference between 31G and PCI groups.

### Discussion

Injections using the PCI needle were simple, easy to perform, and compared to the 31G needle, resulted in less leakage, less variability, and fewer AC injections; all parameters important for gene therapy to limit off-target tissue exposure of the virus and transgene. Although area of corneal infiltration after injection and GFP expression were similar with the two needle types, the PCI needle decreased drug dose variability, increased target tissue drug levels, and provided a simple method for dosing.

### References for Example 2

1. Hippert C, Ibanes S, Serratrice N, et al. Corneal transduction by intra-stromal injection of AAV vectors in vivo in the mouse and Ex vivo in human explants. PLoS One. 2012. doi:10.1371/journal.pone.0035318
2. Mohan R R, Schultz G S, Hong J W, Mohan R R, Wilson S E. Gene transfer into rabbit keratocytes using AAV and lipid-mediated plasmid DNA vectors with a lamellar flap for stromal access. Exp Eye Res. 2003; 76(3):373-383. doi:10.1016/S0014-4835(02)00275-0
3. Kamata Y, Okuyama T. Kosuga M, et al. Adenovirus-mediated gene therapy for corneal clouding in mice with mucopolysaccharidosis type VII. Mol Ther. 2001. doi:10.1006/mthe.2001.0461
4. Bemelmans A, Arsenijevic Y, Majo F. Efficient lentiviral gene transfer into corneal stroma cells using a femtosecond laser. Gene Ther. 2009; 16(7):933-938. doi:10.1038/gt.2009.41
5. Mohan R R, Sharma A, Cebulko T C, Tandon A. Vector delivery technique affects gene transfer in the cornea in vivo. Mol Vis. 2010; 16(October):2494-2501.
6. Netto M V, Mohan R R, Ambrósio R, Hutcheon A E K, Zieske J D, Wilson S E. Wound healing in the cornea: a review of refractive surgery complications and new prospects for therapy. Cornea. 2005; 24(5):509-522.
7. Johnson J S, Samulski R J. Enhancement of Adeno-Associated Virus Infection by Mobilizing Capsids into and Out of the Nucleolus. J Virol. 2009. doi:10, 128/JVI.02309-08
8. Tsai M. Chen S, Chou P. Wen L, Tsai R J, Tsao Y. Inducible Adeno-Associated Virus Vector—Delivered Transgene Expression in Corneal Endothelium. 2016; 43(3).

### Example 3

### Voriconazole Ocular Tissue and Fluid Concentrations

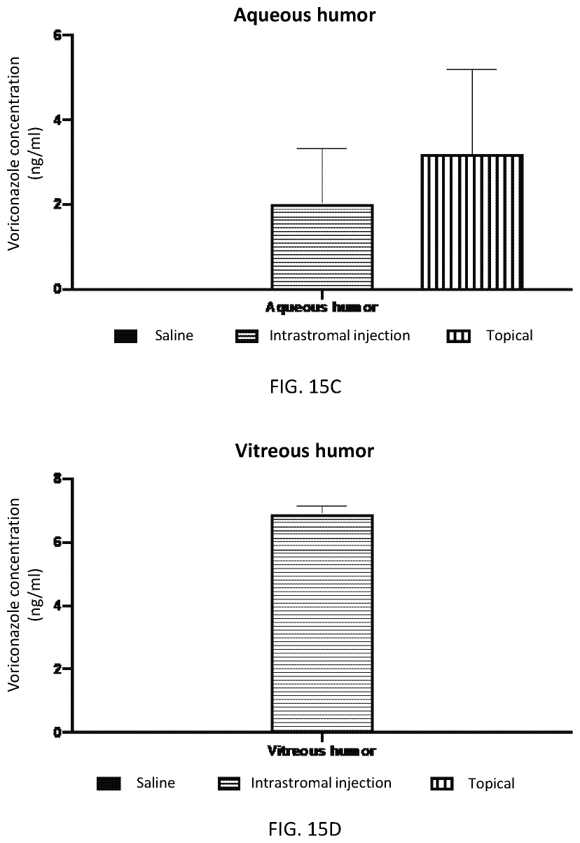

The following tissues/fluids were analyzed for voriconazole concentration following application of either 500 μg of topical voriconazole (divided into 4 doses, given every 6 hours), a single intrastromal injection of 500 μg voriconazole (using a PCI needle, 34 gauge (g), 250 μm length needle) or a single Intrastromal injection of saline in normal New Zealand white rabbits:
Plasma, tears (FIG. 15A), conjunctiva (FIG. 15B), cornea, aqueous humor (FIG. 15C), iris/ciliary body, vitreous humor (FIG. 15D), and retina/choroid.

Samples were collected 6 hours following the last topical dose or 24 hours after the intrastromal injection. Sample size was 6 per tissue per application type.

Concentration of voriconazole was below quantification limits (BQL=5 ng/ml) in all samples dosed with saline, all plasma samples, and all retina/choroidal tissues. There was no significant difference in voriconazole concentrations in conjunctiva (FIG. 15B), cornea, and aqueous humor (FIG. 15C) among treatment groups. In the tear film (FIG. 15A), topical application resulted in significantly higher voriconazole concentration compared to saline treated (P=0.04), but was not significant than intrastromal concentrations. Vitreous humor voriconazole concentrations were highly significantly greater (P<0.0001) following intrastromal application compared to concentrations following topical voriconazole or saline applications (FIG. 15D).

FIG. 15A shows topical significantly greater than saline (P=0.04), but no significant difference between intrastromal and topical, in tears. FIG. 15B shows no significant difference between saline, intrastromal, and topical in conjunctiva. FIG. 15C shows no significant difference between saline, intrastromal, and topical in aqueous humor. FIG. 15D shows intrastromal significantly greater than saline and topical (P<0.0001) in vitreous humor.

### CONCLUSIONS

Low levels of voriconazole were measured in ocular surface and intraocular tissues 24 hours following either topical or intrastromal application of 500 μg of voriconazole. Voriconazole levels following a single intrastromal injection were comparable to concentrations achieved after topical administration, however, intrastromal administration resulted in a significantly greater concentration in the vitreous compared to topical dosing. his suggests that intrastromal injection results in a better intraocular penetration and drug levels compared to topical administration of voriconazole.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A device configured for selectively contacting a target region of the eye, the device comprising:
a shaft, the shaft having a first end and a second opposite end having a tip, optionally with a hollow pathway disposed between the first end and the second end, wherein the tip has a dimension configured for selectively contacting the target region of the eye; and
a hub that houses the shaft, the hub having a proximal end and a distal end.

2. The device of claim 1, wherein the tip comprises a bevel having a dimension configured for selectively contacting the target region of the eye.

3. The device of claim 1 or claim 2, wherein the hub comprises a connector at the proximal end for connecting to a delivery device.

4. The device of any one of claims 1-3, wherein the target region of the eye is selected from the group consisting of the cornea, an ocular anterior segment, the conjunctiva, an anterior chamber, iridocorneal angle, trabecular meshwork, sclera, subretinal space, choroid, and Schlem's canal.

5. The device of any one of claims 1-4, wherein the hub has a predetermined shape configured based on the target region of the eye, optionally wherein the hub shape comprises a bullnose, further optionally wherein the bullnose has a radius of curvature of about 0.2 mm to about 1.5 mm or optionally wherein the hub shape comprises a conical shape, further optionally wherein the conical shape comprises a cone angle ranging between about 45 and about 150 degrees.

6. The device of any one of claims 1-5, wherein the hub comprises a polymer, optionally wherein the polymer is selected from the group consisting of polypropylene (PP), polyethylene (PE), polyether ether ketone (PEEK), acrylonitrile butadiene styrene (ABS).

7. The device of any one of claims 1-6, wherein the hub comprises a surface, wherein the surface comprises or is coated by biocompatible elastomer, optionally wherein the biocompatible polymer comprises a silicone.

8. The device of any one of claims 1-7, further comprising a stop region disposed at the distal end of the hub such that only the tip of the shaft extends beyond the stop region.

9. The device of claim 8, wherein the stop region comprises a biocompatible elastomeric material, optionally wherein the elastomeric material comprises silicone.

10. The device of any one of claims 1-9, wherein the shaft comprises a metal, optionally wherein the metal is stainless steel, further optionally wherein the stainless steel is an alloy 304 or 310 material.

11. The device of any one of claims 1-10, wherein the target region of the eye has a thickness and the dimension of the tip and/or bevel comprises a length, wherein the length of the tip and/or the length of the bevel is less than the thickness of the target region of the eye, optionally wherein the target region is the cornea and the length of the tip and/or the length of the bevel is less than the thickness of the cornea, further optionally wherein the length of the tip ranges from about 0.1 to about 1 mm.

12. The device of any one of claims 1-11, wherein the length of the bevel is less than the length of the tip, optionally wherein the length of the bevel ranges from about 0.1 to about 0.8 mm.

13. The device of any one of claims 1-12, wherein the dimension of the tip comprises a gauge and the gauge of the tip ranges from about 30 gauge to about 40 gauge.

14. The device of any one of claims 1-13, wherein the tip comprises an opening and the device is configured to deliver an injection volume, optionally wherein the injection volume ranges from about 1 to about 200 microliters.

15. The device of any one of claims 1-14, wherein a configuration of the device for selectively contacting a target region of the eye varies based on a patient species, an intended agent to be delivered, and/or an intended tissue target in the target region.

16. The device of any one of claims 1-15, further comprising a syringe connected to the hub, optionally wherein the syringe comprises a syringe pump, further optionally wherein the syringe comprises a reservoir containing an agent to be delivered.

17. A system for selectively contacting a target region of the eye, comprising the device of any one of claims 1-16.

18. A method for selectively contacting a target region in the eye of a subject, the method comprising:
providing a device according to any one of claims 1-16; and
contacting the target region of the eye with the device.

19. The method of claim 18, wherein the contacting comprises delivering an agent to the target region of the eye and/or wherein the contacting comprises facilitating a surgery on the target region of the eye.

20. The method of claim 18 or claim 19, wherein the agent comprises a fluid or a powder.

21. The method of any one of claims 18-20, wherein the agent comprises a therapeutic agent, an imaging agent, an implant, an ink, and/or a surgical enhancement.

22. The method of claim 21, wherein the therapeutic agent is selected from the group consisting of a topical ocular medication, optionally wherein the topical ocular medication is selected from the group consisting of a steroid, an antibiotic, a NSAID, and an anti-glaucoma agent; a gene therapy vector, optionally wherein the gene therapy vector comprises a virus; a stem cell; and combinations thereof.

23. The method of claim 21, wherein the imaging agent comprises gadolinium.

24. The method of claim 21, wherein the implant comprises a micro-electronic, optionally wherein the micro-electronic comprises a visual aide or an intraocular pressure gauge.

25. The method of claim 21, wherein the ink comprises an ink for cosmetic or therapeutic tattooing.

26. The method of claim 21, wherein the surgical enhancement is a viscoelastic and/or a surgical enhancement for corneal surgery or Lasik surgery.

27. The method of any one of claims 18-26, wherein the target region of the eye is selected from the group consisting of the cornea, an ocular anterior segment, the conjunctiva, an anterior chamber, iridocorneal angle, trabecular meshwork, sclera, subretinal space, choroid, and Schlem's canal.

28. The method of any one of claims 18-27, wherein the target region comprises a diseased and/or injured region of the eye, optionally wherein the diseased and/or injured region comprises an infected region of the eye, further optionally wherein the diseased and/or injured region is in the cornea.

29. The method of any one of claims 18-28, wherein the contacting comprises treating a disease selected from the group consisting of an infection, optionally wherein the infection is a corneal stromal infection; an immune-mediated disease; a genetic disease, optionally wherein the genetic disease is MPS-1; a neovascular disease; and a degenerative disease.

30. The method of any one of claims 18-29, wherein the contacting comprises improving drug penetration or directly providing therapy for an ocular anterior segment disease and/or injury, optionally wherein the ocular anterior segment disease and/or injury is uveitis, glaucoma, and/or trauma.

31. The method of any one of claims 18-30, wherein the contacting comprises positioning the device perpendicular to the target region in the eye of the subject.

32. The method of any one of claims 18-31, wherein the device is used in conjunction with an imaging technique, optionally wherein the imaging technique employs an optical coherence tomography device or a high frequency ultrasound, such that a characteristic of the target region to be contacted is determined and a device having a desired configuration is prepared and/or selected to reach the target region, optionally without passing through the target region.

33. The method of claim 32, wherein the imaging technique determines the thickness and/or depth of the target region and allows the selection of an appropriately configured device to reach the target region.

34. A kit of parts for selectively contacting a target region of the eye, the kit of parts comprising one or more device of any one of claims 1-16 and a container for the one or more device.
